(19) United States
(12) Patent Application Publication
(10) Pub. No.: US 2009/0118202 A1
(43) Pub. Date: May 7, 2009

(54) COMPOSITIONS AND METHODS OF TREATING HYPERTENSION WITH TANNIN COMPLEXES

(75) Inventor: Thomas J. Thekkumkara, Amarillo, TX (US)

Correspondence Address: CHALKER FLORES, LLP, 2711 LBJ FRWY, Suite 1036, DALLAS, TX, 75234, US

(73) Assignee: Texas Tech University, Lubbock, TX (US)

(21) Appl. No.: 12/263,197

(22) Filed: Oct. 31, 2008

Related U.S. Application Data
(60) Provisional application No. 60/984,265, filed on Oct. 31, 2007.

(51) Int. Cl. A61K 31/70; A61P 9/12

(52) U.S. Cl. 514/25

(57) ABSTRACT

The present invention includes methods and compositions to ameliorate one or more symptom of hypertension through the inhibitor of an AT1 receptor by transcriptional down regulation of an angiotensin II type 1 receptor. The composition includes an effective amount of one or more tannic acids disposed in a pharmaceutically acceptable carrier.

### CROSS-REFERENCE TO RELATED APPLICATIONS

This application claims priority to U.S. Provisional Application Ser. No. 60/984,265, filed Oct. 31, 2007, the contents of which is incorporated by reference herein in its entirety.

### STATEMENT OF FEDERALLY FUNDED RESEARCH

This invention was made with U.S. Government support under Contract No. DK072140 awarded by the NIH. The government has certain rights in this invention.

### TECHNICAL FIELD OF THE INVENTION

The present invention relates in general to the field of the treatment of hypertension and more specifically to compositions and methods for to inhibiting AT1 receptor function by transcriptional down regulation of the AT1 receptor to ameliorate one or more symptom of hypertension.

### BACKGROUND OF THE INVENTION

Without limiting the scope of the invention, its background is described in connection with methods and pharmaceutical compositions to ameliorate one or more symptom of hypertension in a subject.

Hypertension commonly referred to as “high blood pressure” is a prevalent disease in many countries around the world. This medical condition can be the result of a variety of factors and lead to potentially life threatening conditions, e.g., strokes, heart attacks, heart failure and arterial aneurysm and is a leading cause of chronic renal failure. In addition, even moderate elevations in arterial blood pressure can lead to a dramatically shortened life expectancy.

Currently there are numerous drugs therapies used to decrease arterial pressure and control the morbidity of hypertension; however, some side effects have been observed during clinical applications of these drugs when administered to patients for a long period. In addition, it is necessary to find new and alternative antihypertensive drugs provide clinical physicians more choices of drugs for treatment without greatly inducing side effects.

Some hypertension treatments are prepared from natural plants or animals; however, many contain ineffective compounds in addition to the effective compound making them unacceptable treatments by western medicine standards. Therefore, the effective components must be purified the so that their actual treating effect the can be experimented and verified.

One system that helps regulate long-term blood pressure and extracellular volume in the body is the Renin-Angiotensin System (RAS) and is often manipulated clinically to treat high blood pressure, e.g., inhibitors of angiotensin-converting enzyme (ACE inhibitors) are often used to reduce the formation of angiotensin II and Angiotensin Receptor Blockers (ARBs) are used to prevent angiotensin II from acting on angiotensin receptors. Generally, renin-angiotensin system includes a class of G protein-coupled receptors (e.g., angiotensin receptors) responsible for the signal transduction of the main effector hormone angiotensin II.

One method of treating high blood pressure includes the inhibition of ACE by procyanidins (i.e., a type of condensed tannin) has been described in the literature [1] to inhibit the AT1 receptor indirectly by inhibiting the conversion of angiotensin I to angiotensin II using the same mechanism used by typical ACE inhibitors, e.g., captopril or enalapril.

### SUMMARY OF THE INVENTION

The present inventor recognized that patients who are on ACE inhibitor can still have high blood pressure due to the conversion of AngI to AngII by proteases (e.g., chymase) and other factors. Those patients who are on ARB if they miss the drug regiment there will be significant blood pressure due to over accumulation of AngII. However inhibiting the AT1 receptor gene transcription will shut down the receptor function completely due to down-regulation of receptor protein expression.

The present inventors were the first to recognize that tannic acid may be used to ameliorate one or more symptom of hypertension through the inhibition of AT1 receptor function by transcriptional down regulation of the AT1 receptor.

The present inventors recognized that tannic acid does not function like condensed tannin and provides a novel method of inhibition of AT1 receptor by transcriptional down regulation of the AT1 receptor. Until the present inventors discovered that tannic acid and other hydrolysable tannins could inhibit AT1 receptor function by transcriptional down regulation, the skilled artisan had no indication that it may be used to treat hypertension.

The present invention provides the use of tannic acid as a specific inhibitor for the angiotensin II type 1 (AT1) receptor through decreasing the levels of AT1 receptor expression by inhibiting gene transcription. The present invention provides an inhibition that is specific and reversible and does not affect the affinity of angiotensin II binding to the AT1 receptor. The present invention provides pharmaceutical compositions containing tannic acid to function as an antihypertensive agent.

The present invention provides a pharmaceutical composition for treating hypertension. The composition includes an effective amount of one or more tannic acids disposed in a pharmaceutically acceptable carrier. The one or more tannic acids to ameliorate one or more symptom of hypertension through the inhibitor of an AT1 receptor function by transcriptional down regulation of an angiotensin II type 1 receptor.

In addition the present invention provides a method of modulating an AT1 receptor by administering a pharmaceutical composition having an effective amount of one or more tannic acids disposed in a pharmaceutically acceptable carrier to a subject. The one or more tannic acids inhibit an AT1 receptor function by transcriptional down regulation of an angiotensin II type 1 receptor.

The present invention also provides a pharmaceutical composition for modulating an AT1 receptor. The pharmaceutical composition includes an effective amount of one or more tannic acids disposed in a pharmaceutically acceptable carrier. The one or more tannic acids inhibit an AT1 receptor function by transcriptional down regulation of an angiotensin II type 1 receptor.

The present invention also provides a method of treating hypertension in a subject by administering to a subject suspected to need of an anti-hypertension treatment. The pharmaceutical composition includes an effective amount of one or more tannic acids disposed in a pharmaceutically acceptable carrier. The one or more tannic acids inhibit an AT1 receptor function by transcriptional down regulation of an angiotensin II type 1 receptor.

### BRIEF DESCRIPTION OF THE DRAWINGS

For a more complete understanding of the features and advantages of the present invention, reference is now made to the detailed description of the invention along with the accompanying figures and in which:

FIG. 1 is an image that illustrates the structure of the constitutes of tannic acid;

FIG. 2 is an image that illustrates the structure of tannic acid;

FIG. 3A is an image of a plot that illustrates the effects of tannic acid on native AT1, and FIG. 3B is an image of a graph illustrating specific AngII binding with exposure to tannic acid for various times;

FIGS. 4A and 4B are plots illustrating competition binding studies to determine the AT1 receptor affinity in WB cells, performed on cells treated with and without tannic acid;

FIG. 5 is an image of a graph illustrating the effects of tannic acid on MAP-kinase activity and receptor expression;

FIGS. 6A-6C are images of Western blots of rat liver cells exposed to tannic acid in the presence or absence of a MAP-Kinase specific cell permeable inhibitor and immunoblotted with various antibody;

FIGS. 7A and 7B are images of graphs that illustrates AT1 specific AngII binding and the effects of tannic acid on AT1 receptor with constitutively active promoter; and

FIG. 8 is an image that illustrates control actions of angiotensin II.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Although, many agonists (e.g., angiotensin II, growth factors, low-density lipoprotein, cholesterol, insulin, glucose, estrogen, progesterone, reactive oxygen species, cytokines, nitric oxide, and many others) are known to regulate AT1-receptor expression in vascular cells, the present inventors were the first to recognize that tannic acid compositions may be used to transcriptional down-regulate the AT1 receptor to ameliorate one or more symptom of hypertension.

The inhibition of ACE by procyanidins (i.e., a type of condensed tannin) has been described in the literature [1]; however, these condensed tannins inhibit the AT1 receptor indirectly using the same mechanism as typical ACE inhibitors (e.g., captopril or enalapril) through the inhibition of the conversion of angiotensin I to angiotensin II. Similarly, other references disclose the inhibition of the AT1 receptor by tannins [2]; however, the tannins disclosed are proanthocyanidins (e.g., condensed tannins, not hydrolysable tannins such as tannic acid) that inhibit the AT1 receptor through direct binding. Tannic acid has used to prevent metal corrosion, as an acidic exfoliating agent in dermatological and cosmetic preparations, as a reagent in microscopy, as an active agent for cancer treatment, or as a component of oral medicinal preparations, see U.S. Pat. Nos.: 5,773,419, 6,063,770, 6,133,311, 6,187,315, 6,200,568, 6,207,694, 6,210,681, 6,245,336, 6,271,246, 6,383,523, 6,440,471, 6,469,053, and 6,696,485, and United States Application Publication Numbers 20020040005, 20020009423, 20010021398, 20020028260, 20040047945, 20030068792, and 20060264375.

The present invention provides for the amelioration of one or more symptom of hypertension by using a pharmaceutical composition having tannic acid or other hydrolysable tannins that inhibit AT1 receptor function by transcriptional down regulation of the AT1 receptor.

Angiotensin II is an effector peptide in the Renin Angiotensin System (RAS) and has far-reaching effects on vascular structure, growth, and fibrosis, and is a key regulator of hypertension, vascular remodeling, and inflammation. Treatments that block the pathologic effects of the RAS at several points have been shown to limit target-organ damage in hypertension and to decrease cardiovascular morbidity and mortality. Previous studies have shown that plant-derived polyphenols appear to provide protection from hypertension and cardiovascular diseases.

The present inventors recognized that tannins compositions provide protection from hypertension and cardiovascular diseases. In general, tannins are astringent, bitter-tasting plant polyphenols characterized as being oligomeric compounds with multiple structure units having free phenolic groups. The molecular weights can range from a few hundred to tens of thousands. Tannins may be subdivided into two groups, e.g., hydrolyzable tannins and proanthocyanidins (i.e., condensed tannins) based on the base structure of the molecule. For example, the present invention includes hydrolyzable tannin obtained from oak gall nuts from Quercus infectoria. Oak gall nuts (also known as galls) are tumors formed by fungi, insects or bacteria on oak trees and are a rich source of tannic acid. The skilled artisan will recognize that other trees of trees may be used to produce tannins for use in the present invention.

Generally, the condensed tannins are widely distributed oligomers or polymers of flavonoid units (i.e. flavan-3-ol) linked by carbon-carbon bonds that not susceptible to cleavage by hydrolysis. In contrast, hydrolyzable tannins are molecules with a polyol as a central core (e.g., generally D-glucose) with the hydroxyl groups at least partially esterified with phenolic groups like gallic acid (e.g., gallotannins) or ellagic acid (e.g., ellagitannins). At least 3 hydroxyl groups of the glucose must be esterified to exhibit a sufficiently strong binding capacity to be classified as a tannin. The physical properties of hydrolyzable tannins also differ from the nonhydrolyzable tannins, e.g., hydrolyzable tannins may be hydrolyzed by hot water, enzymes, mild acids or mild bases to yield carbohydrate and phenolic acids; whereas condensed tannins would not be hydrolyzed under the same conditions.

The present invention provides the use of hydrolysable tannic acids to affect angiotensin type 1 (AT1) receptor expression. The present invention used WB (i.e., a continuously passaged rat liver epithelial cell line) which expresses the native AT1 receptor. Under normal culture conditions, cells were exposed to tannic acid and changes in receptor expression and signaling intermediates were determined. Exposure of cells to tannic acid resulted in the down regulation of AT1 receptor binding in a concentration (over the range of about 12.5 to about 100 μg/ml) and time (over the range of between about 2 and about 20 hours) dependent manner with no change in affinity of the receptor. The tannic acid of the present invention provides an inhibitory effect on the AT1 receptor in a specific and reversible manner.

Under similar conditions, increased phosphorylation of extracellular signal regulated kinase (ERK) or MAP-kinases p42 and p44 was observed. Pretreatment of cells with MAP kinase specific inhibitor was ineffective in preventing tannic acid induced MAP-kinases phosphorylation and down regulation of the AT1 receptor indicating that the observed inhibitory effect of tannic acid effect on AT1 receptor is mediated through MAP-kinases signaling intermediate(s).

The present invention provides a dietary polyphenol that blunt hypertension, at least in part through inhibition of AT1 receptor. The tannic acid of the present invention mediates activation of MAP kinase(s) and AT1 receptor down regulation and inhibits the AT1 receptor expression.

Although some hydrolysable tannins are known, the tannins as a group are complex and varied as a result of the source from which they are derived, e.g., ellagitannins corilagin and geraniin. Commercial tannic acid is comprised of mixtures of hydrolysable tannins (e.g., mostly gallotannins) from multiple sources and are very different from source to source [3]. Similarly, gallotannins 6-O-galloyl-D-glucose and 1, 2, 3, 4, 6-penta-O-galloyl-β-D-glucose, are also known. Although commercial sources provide a nominal molecular weight for tannic acid, the preparations are heterogenous mixtures of galloyl esters, and one the components is 1, 2, 3, 4, 6-penta-O-galloyl-β-D-glucose. Therefore, tannic acid is basically a plant extract that can be derived from a variety of sources and as such, can have different compositions depending on that source. For example, U.S. Pat. No. 5,266,319 entitled “Tannin derivatives and their use for treatment of hypertension” discloses a treatment of hypertension with an effective amount of tannins extracted from Sapium sebiferum leaves, the tannins are 6-O-galloyl-D-glucose, corilagin, geraniin and 1, 2, 3, 4, 6-penta-O-galloyl-β-D-glucose. The treatment involved the inhibition of the AT1 receptor by either direct binding of a blocker to a binding site in the AT1 receptor (e.g. Angiotensin Receptor Blockers or ARBs) and/or the indirect inhibition through modulation of the enzymatic activity of Angiotensin Converting Enzyme (ACE) by different ligands.

The present inventors discovered a source of tannic acid that provides specific inhibitor for the angiotensin II type 1 (AT1) receptor through decreasing the levels of AT1 receptor expression by inhibiting gene transcription. The present invention provides pharmaceutical compositions containing tannic acid to function as an antihypertensive agent. The present inventor provides a tannic acid composition and a method of affecting an AT1 receptor.

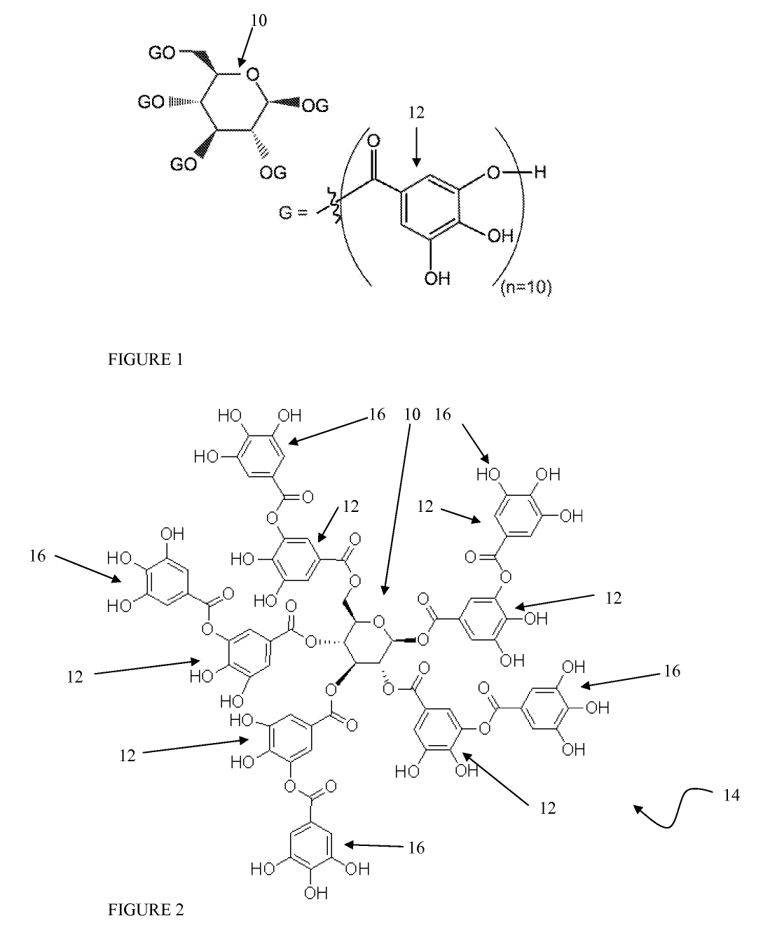

FIG. 1 is an image that illustrates the structure of the tannic acid constitutes. The tannic acid includes a glucose core 10, which covalently links to 3-10 gallic acid residues 12 through hydrolyzable ester bonds. In addition, each gallic acid residue 12 can covalently link to other gallic acid molecules (not shown) and include modifications and substitutions to both the core and the gallic acid residues.

FIG. 2 is an image that illustrating the tannic acid molecule 14 which includes a glucose core 10 covalently links to 10 gallic acid residues 12 through hydrolyzable ester bonds. In addition, each gallic acid residue 12 is covalently link to a second gallic acid molecule 16. in addition the tannic acid molecule 14 may include a third gallic acid residues not shown attached to one or more of the gallic acid residues 12. Furthermore, the glucose core 10 and/or the gallic acid residues 12 may individually include modifications and substitutions.

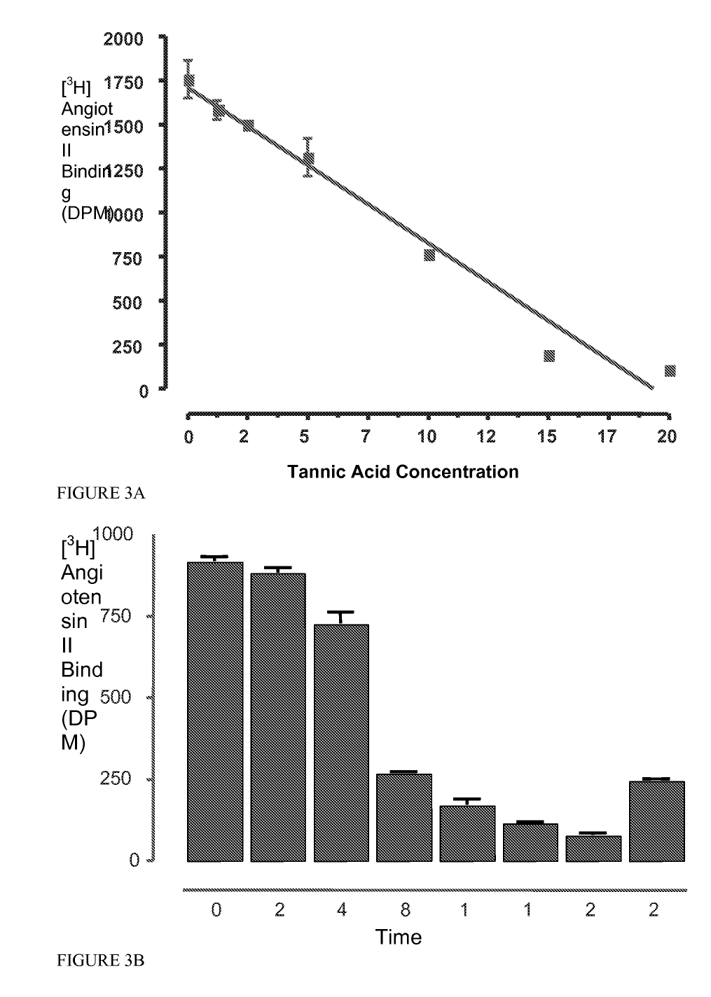

FIG. 3A is an image of a plot illustrating the effects of tannic acid on native AT1, rat liver cells were exposed to tannic acid at indicated concentration for 20 hours. FIG. 3B is an image of a graph illustrating the exposure to tannic acid (100 μg/ml) for indicated times and AT1 specific AngII binding measured. AT1 specific binding is defined as that portion of the total binding displaced by 1 μM unlabeled angiotensin II, where the data are expressed as mean±SEM.

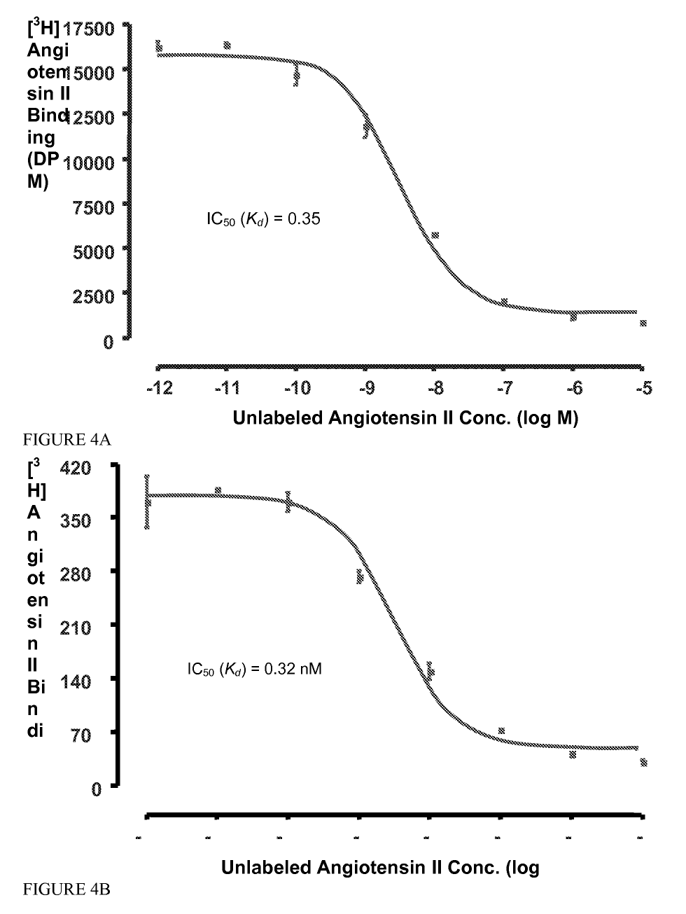

FIG. 4A is an image of a plot illustrating the AT1 receptor affinity in WB cells, competition binding studies were performed on cells treated without tannic acid and with tannic acid in FIG. 4B. Cultures of confluent cells were incubated with 50 pM [3H]AngII for about 60 minutes at 22° C. in the presence of indicated concentrations of unlabeled AngII. Each point represents the mean±SEM of triplicate determinations. Nonlinear least squares regression analysis gave an IC50 (Kd) of 0.35 nM for receptors in cells exposed to normal medium as seen in FIG. 4A and an IC50 (Kd) of 0.32 nM for receptors in cells exposed to tannic acid as seen in FIG. 4B.

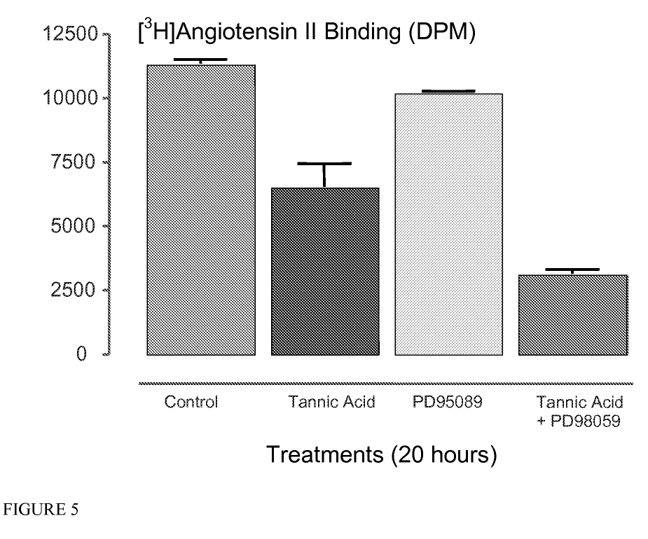

FIG. 5 is an image of a graph illustrating the effects of tannic acid on MAP-kinase activity and receptor expression, binding studies were performed on cells treated with tannic acid in the presence or absence of a MAP-Kinase specific cell permeable inhibitor (e.g., PD98059) for 20 hours and AT1 specific AngII binding measured. AT1 specific binding is defined as that portion of the total binding displaced by 1 μM unlabeled angiotensin II, where the data are expressed as mean±SEM.

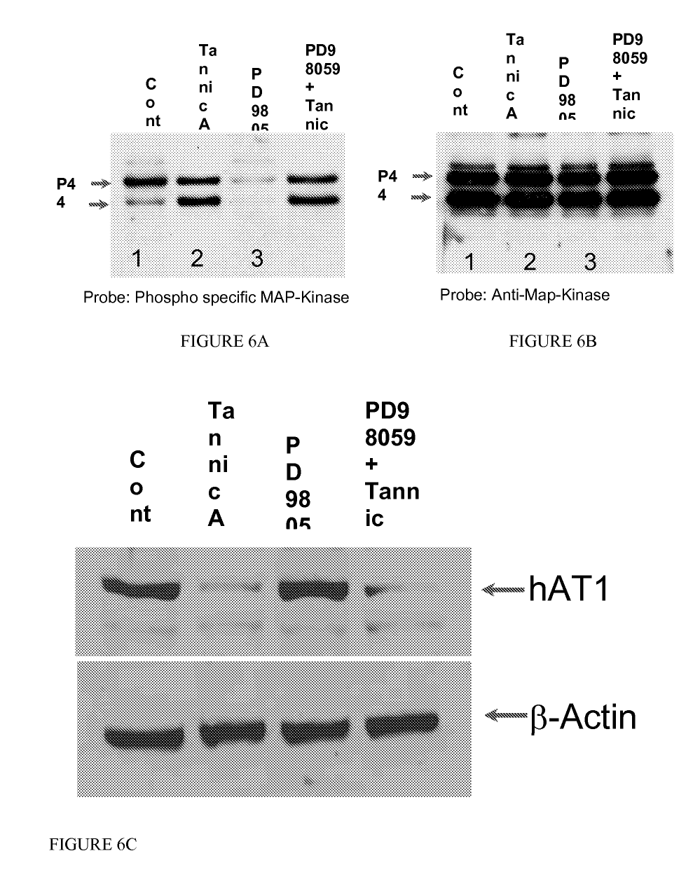

FIGS. 6A-6C are images of a Western blot illustrating rat liver cells exposed to tannic acid in the presence or absence of the MAP-Kinase specific cell permeable inhibitor PD 98059 for 20 hours and total cell extracts were prepared. Equal amount of proteins were resolved by 8% polyacrylamide gel electrophoresis and immunoblotted with phospho-specific MAP-Kinase antibody in FIG. 6A, anti-MAP-kinase antibody in FIG. 6B, AT1 specific antibody in FIG. 6C top or anti-beta-actin antibody in FIG. 6C bottom. Immunoreactive bands were visualized using a chemiluminescence Western blotting system according to the manufacturer's instruction. In each plate protein concentration was determined using Bio-Rad protein assay reagent.

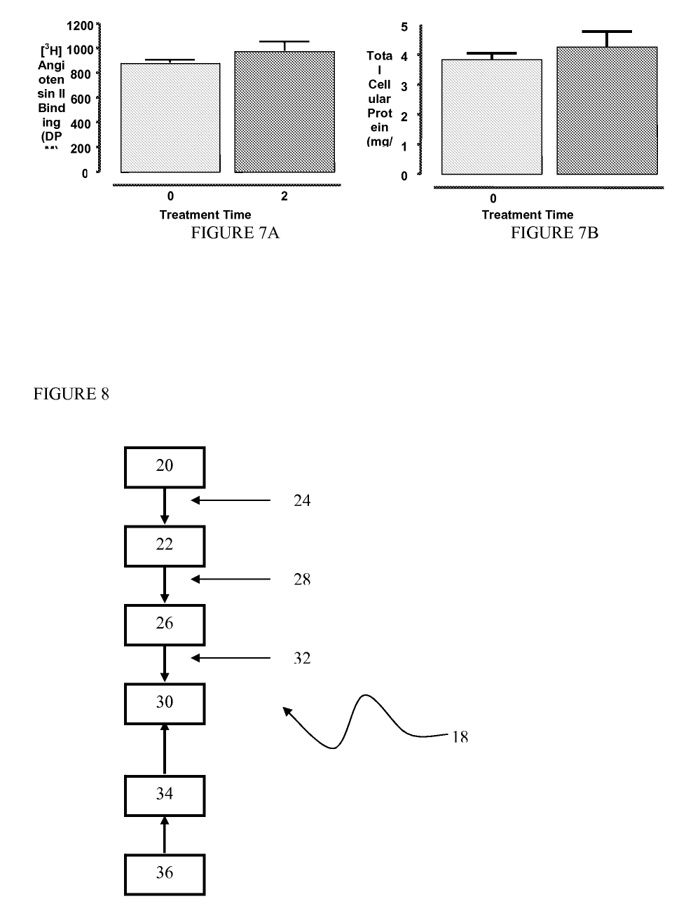

FIGS. 7A and 7B are images of graphs illustrating the effects of tannic acid on AT1 receptor with constitutively active promoter (CMV), receptor transfected cells were exposed to tannic acid (100 μg/ml) for indicated times and AT1 specific AngII binding measured. AT1 specific binding is defined as the portion of the total binding displaced by 1 μM unlabeled angiotensin II, where the data are expressed as mean±SEM in FIG. 7A. In each plate protein concentration was determined using Bio-Rad protein assay reagent in FIG. 7B.

FIG. 8 is an image illustrating the control actions of angiotensin II. The conversion of angiotensinogen 20 to angiotensin I 22 is affected by the RNI system 24. The angiotensin I 22 to the angiotensin II 26 is affected by ACE 28, which in turn affects the angiotensin Type 1 Receptor 30. Current Drugs that affect ARBs include Candesartan, Losartan, and Valsartan. Current Drugs that affect ACE inhibitors include Captopril, Enalapril, Ramipril. In addition, the angiotensin Type 1 Receptor 30 is also affected by a receptor mRNA, under control by the receptor gene through a gene repressor, e.g., tannic acid.

It is contemplated that any embodiment discussed in this specification can be implemented with respect to any method, kit, reagent, or composition of the invention, and vice versa. Furthermore, compositions of the invention can be used to achieve methods of the invention.

The present invention provides a pharmaceutical composition for treating hypertension. The composition includes an effective amount of one or more tannic acids disposed in a pharmaceutically acceptable carrier. The one or more tannic acids to ameliorate one or more symptom of hypertension through the inhibitor of an ACE receptor by transcriptional down-regulation of an angiotensin II type 1 receptor. The carrier used in the present invention may be a substance, whether biodegradable or not, that is physiologically acceptable for human or animal use and may be pharmacologically active or inactive.

In addition, the present composition may include carrier, anti-adherents, anti-sticking agents, glidants, flow promoters, lubricants, talc, magnesium stearate, fumed silica, micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate, binders, buffering agents, antioxidants, chelating agents, surfactants, colorant, flavorant, sweetening agent, tablet antiadherents, diluent, excipient, opaquant, glidant, lubricant, polishing agent, pharmaceutically acceptable salts and a combination thereof.

The compositions and formulations of the present invention may be formulated into a variety of dosage forms and concentrations and include but not limited to oral administration include, but are not limited to, powders, granules, microparticulates, nanoparticulates, suspensions, solutions in water, non-aqueous solutions, capsules, gel capsules, sachets, tablets, minitablets, emulsions, liposome-containing formulations and combinations thereof. The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Actis-Goretta, L., Ottaviani, J. I., Keen, C. L., and Fraga, C. G. (2003) “Inhibition of angiotensin converting enzyme (ACE) activity by flavan-3-ols and procyanidins”. FEBS Lett. 555(3), 597-600.
Caballero-George, C., Vanderheyden, P. M., De Bruyne, T., Shahat, A. A., Van den Heuvel, H., Solis, P. N., Gupta, M. P., Claeys, M., Pieters, L., Vauquelin, G., and Vlietinck, A. J. (2002) “In vitro inhibition of [³H]-angiotensin binding on the human AT1 receptor by proanthocyanidins from Guazuma ulmifolia bark”. Planta Med. 68(12), 1066-71.
Makkar, H. P. S. and Becker, K. (1993) “Behaviour of tannic acid from various commercial sources towards redox, metal complexing and protein precipitation assays of tannins”. Journal of the Science of Food and Agriculture 62(3), 295-99.
Liu, J. C. et al. (2003) “Antihypertensive effects of tannins isolated from traditional Chinese herbs as non-specific inhibitors of angiotensin converting enzyme.” Life Sci. 73(12), 1543-55.
U.S. Pat. No. 5,773,419 entitled, Method of treating cancer with tannic acid; U.S. Pat. No. 6,063,770 entitled, Tannic acid compositions for treating cancer; U.S. Pat. No. 6,133,311 entitled, Method for preventing or treating elevated blood lipid level-related diseases by administering natural phenolic compounds; U.S. Pat. No. 6,187,315 entitled, Compositions and methods of treating cancer with tannin complexes; U.S. Pat. No. 6,200,568 entitled, Composition and method of treating cancer with tannic acid and tannin complexes; U.S. Pat. No. 6,207,694 entitled, Pharmaceutical compositions and methods for managing scalp conditions; U.S. Pat. No. 6,210,681 entitled, Plant proanthocyanidin extracts; U.S. Pat. No. 6,245,336 entitled, Prevention and treatment of acetaminophen toxicity with grape seed proanthocyanidin extract; U.S. Pat. No. 6,271,246 entitled, Pharmaceutical compositions for managing scalp conditions; U.S. Pat. No. 6,383,523 entitled, Pharmaceutical compositions and methods for managing skin conditions; U.S. Pat. No. 6,440,471 entitled, Plant proanthocyanidin extracts; U.S. Pat. No. 6,469,053 entitled, Use of procyanidins in the maintenance of vascular health and modulation of the inflammatory response; U.S. Pat. No. 6,696,485 entitled, Procyanidin and cyclo-oxygenase modulator compositions; United States Patent Application Publication number 20020040005 entitled, Composition and method of treating cancer; United States Patent Application Publication number 20020009423 entitled, Methods for managing scalp conditions; United States Patent Application Publication number 20010021398 entitled, Plant proanthocyanidin extracts; United States Patent Application Publication number 20020028260 entitled, Plant proanthocyanidin extracts; United States Patent Application Publication number 20040047945 entitled, Use of tannins and polymers to regulate digestion in animals; United States Patent Application Publication number 20030068792 entitled, Targeted enzymes; and United States Patent Application Publication number 20060264375 entitled, Elastin protective polyphenolics and methods of using the same.

What is claimed is:

1. A pharmaceutical composition for treating hypertension comprising:
an effective amount of one or more tannic acids disposed in a pharmaceutically acceptable carrier, wherein the one or more tannic acids to ameliorate one or more symptom of hypertension through the inhibitor of an AT1 receptor by transcriptional down regulation of a angiotensin II type 1 receptor.

2. The composition of claim 1, wherein the one or more tannic acids comprise an extract derived from Quercus infectoria.

3. The composition of claim 1, wherein the one or more tannic acids comprise a O-galloyl-β-D-glucose core with 6 or more O-galloyl subunits.

4. The composition of claim 1, wherein the one or more tannic acids comprise 1,1,2,2,3,3,4,4,6,6-deca-O-galloyl-β-D-glucose.

5. The composition of claim 1, wherein the pharmaceutical composition further comprises one or more selected from the group of carriers, anti-adherents, anti-sticking agents, glidants, flow promoters, lubricants, talcs, magnesium stearate, fumed silicas, micronized silicas, polyethylene glycols, surfactants, waxes, stearic acid, stearic acid salts, stearic acid derivatives, starchs, hydrogenated vegetable oils, sodium benzoate, sodium acetate, leucine, PEG-4000, magnesium lauryl sulfate, binders, buffering agents, antioxidants, chelating agents, surfactants, colorants, flavorants, sweetening agents, tablet antiadherents, diluents, excipients, opaquants, glidants, lubricants, polishing agents, pharmaceutically acceptable salts and combinations thereof.

6. A pharmaceutical composition for modulating an AT1 receptor comprising:
an effective amount of one or more tannic acids disposed in a pharmaceutically acceptable carrier, wherein the one or more tannic acids inhibit an AT1 receptor by transcriptional down regulation of a angiotensin II type 1 receptor.

7. The composition of claim 6, wherein the one or more tannic acids comprise an extract derived from Quercus infectoria.

8. The composition of claim 6, wherein the one or more tannic acids comprise a O-galloyl-β-D-glucose core with 6 or more O-galloyl subunits.

9. The composition of claim 6, wherein the one or more tannic acids comprise 1,1,2,2,3,3,4,4,6,6-deca-O-galloyl-β-D-glucose.

10. The composition of claim 6, wherein the pharmaceutical composition further comprises one or more selected from the group of carriers, anti-adherents, anti-sticking agents, glidants, flow promoters, lubricants, talcs, magnesium stearate, fumed silicas, micronized silicas, polyethylene glycols, surfactants, waxes, stearic acid, stearic acid salts, stearic acid derivatives, starchs, hydrogenated vegetable oils, sodium benzoate, sodium acetate, leucine, PEG-4000, magnesium lauryl sulfate, binders, buffering agents, antioxidants, chelating agents, surfactants, colorants, flavorants, sweetening agents, tablet antiadherents, diluents, excipients, opaquants, glidants, lubricants, polishing agents, pharmaceutically acceptable salts and combinations thereof.

11. A method of modulating an AT1 receptor comprising the steps of:
administering a pharmaceutical composition comprising an effective amount of one or more tannic acids disposed in a pharmaceutically acceptable carrier to a subject, wherein the one or more tannic acids inhibit an AT1 receptor by transcriptional down regulation of a angiotensin II type 1 receptor.

12. The method of claim 11, wherein the one or more tannic acids comprise an extract derived from Quercus infectoria.

13. The method of claim 11, wherein the one or more tannic acids comprise a O-galloyl-β-D-glucose core with 6 or more O-galloyl subunits.

14. The method of claim 11, wherein the one or more tannic acids comprise 1,1,2,2,3,3,4,4,6,6-deca-O-galloyl-β-D-glucose.

15. The method of claim 11, wherein the pharmaceutical composition further comprises one or more selected from the group of carriers, anti-adherents, anti-sticking agents, glidants, flow promoters, lubricants, talcs, magnesium stearate, fumed silicas, micronized silicas, polyethylene glycols, surfactants, waxes, stearic acid, stearic acid salts, stearic acid derivatives, starchs, hydrogenated vegetable oils, sodium benzoate, sodium acetate, leucine, PEG-4000, magnesium lauryl sulfate, binders, buffering agents, antioxidants, chelating agents, surfactants, colorants, flavorants, sweetening agents, tablet antiadherents, diluents, excipients, opaquants, glidants, lubricants, polishing agents, pharmaceutically acceptable salts and combinations thereof.

16. A method of treating hypertension in a subject comprising the steps of:
administering to a subject suspected to be in need thereof a pharmaceutical composition comprising an effective amount of one or more tannic acids disposed in a pharmaceutically acceptable carrier, wherein the one or more tannic acids inhibit an AT1 receptor by transcriptional down regulation of a angiotensin II type 1 receptor.

17. The method of claim 16, wherein the one or more tannic acids comprise an extract derived from Quercus infectoria.

18. The method of claim 16, wherein the one or more tannic acids comprise a O-galloyl-β-D-glucose core with 6 or more O-galloyl subunits.

19. The method of claim 16, wherein the one or more tannic acids comprise 1,1,2,2,3,3,4,4,6,6-deca-O-galloyl-β-D-glucose.

20. The method of claim 16, wherein the pharmaceutical composition further comprises one or more selected from the group of carriers, anti-adherents, anti-sticking agents, glidants, flow promoters, lubricants, talcs, magnesium stearate, fumed silicas, micronized silicas, polyethylene glycols, surfactants, waxes, stearic acid, stearic acid salts, stearic acid derivatives, starchs, hydrogenated vegetable oils, sodium benzoate, sodium acetate, leucine, PEG-4000, magnesium lauryl sulfate, binders, buffering agents, antioxidants, chelating agents, surfactants, colorants, flavorants, sweetening agents, tablet antiadherents, diluents, excipients, opaquants, glidants, lubricants, polishing agents, pharmaceutically acceptable salts and combinations thereof.
